(19) United States
(12) Patent Application Publication
(10) Pub. No.: US 2002/0192229 A1
(43) Pub. Date: Dec. 19, 2002

(54) MHC class I associated peptides for prevention and treatment of tuberculosis

(76) Inventors: David Flyer, Olney, MD (US); Mark M. Ross, Charlottesville, VA (US); Donald F. Hunt, Charlottesville, VA (US); Forest M. White, Charlottesville, VA (US); Victor H. Engelhard, Charlottesville, VA (US); Ramila Philip, Charlottesville, VA (US)

Correspondence Address: CARELLA, BYRNE, BAIN, GILFILLAN, CECCHI,, STEWART & OLSTEIN, 6 Becker Farm Road, Roseland, NJ, 07068, US

(21) Appl. No.: 10/022,286

(22) Filed: Dec. 13, 2001

Related U.S. Application Data
(60) Provisional application No. 60/264,978, filed on Jan. 30, 2001.
(60) Provisional application No. 60/255,292, filed on Dec. 13, 2000.

(51) Int. Cl. A61K 39/00; C07K 7/08; C07K 7/06

(52) U.S. Cl. 424/185.1; 530/328; 530/327

(57) ABSTRACT

The present invention relates to compositions and methods for the prevention, treatment, and diagnosis of tuberculosis, and discloses peptides, polypeptides, and polynucleotides that can be used to stimulate a CTL response against tuberculosis. The peptide and/or proteins of the invention may be used as a therapeutic drug to stimulate the immune system to recognize and eliminate M. tuberculosis in infected cells or as a vaccine for the prevention of disease. Antibodies that react with the immunogens of the invention, as well as methods of using these antibodies for prevention and treatment of disease, are also disclosed.

This application claims priority of U.S. Provisional Applications 60/264,978, filed Jan. 30, 2001 and 60/255,292, filed Dec. 13, 2000, the disclosures of which are hereby incorporated by reference in their entirety.

A portion of this invention was made using funds provided by the U.S. Government under Grant No. NIH AI-33993 and the U.S. Government may therefore have certain rights in this invention.

### FIELD OF THE INVENTION

The present invention relates generally to the field of immunogens whose structures incorporate peptides derived from Mycobacterium tuberculosis and to methods of using such peptide as a basis for the prevention and treatment of diseases such as tuberculosis.

### BACKGROUND OF THE INVENTION

The mammalian immune system has evolved a variety of mechanisms to protect the host from microorganisms, an important component of this response being mediated by cells referred to as T cells and by antibodies derived from B cells. In combating bacterial infections, antibodies are especially important but likewise are specialized T cells that function primarily by recognizing and killing infected cells. The latter also function by secreting soluble molecules called cytokines that mediate a variety of functions of the immune system.

Thus, the immune system is highly developed to deal with infectious organisms as well as with the elimination of cells infected with such organisms. Among the latter are bacterial infections, such as tuberculosis.

It is estimated that close to a third of the worlds population is infected with Mycobacterium tuberculosis (Mtb). With 8 million new cases and 3 million deaths occurring annually, tuberculosis is a major public health problem in developing countries as well as industrialized countries where a resurgence in tuberculosis is occurring. Studies conducted by the World Health Organization (WHO) have shown that the current vaccine, a live attenuated bacillus Calmette-Guerin (BCG), is only marginally effective, being able to prevent an estimated 5% of all potentially vaccine preventable deaths due to M. tuberculosis. The generation of a new and effective vaccine for tuberculosis is therefore critical in controlling the disease on a worldwide scale.

Cells infected with the tubercle bacillus can be destroyed by the immune system in a process involving lymphocytes, especially cytotoxic T lymphocytes, or CTLs. In order for CTLs to kill infected cells, or secrete cytokines in response to an infected cell, the CTL must first recognize that cell as being infected. This process involves the interaction of the T cell receptor, located on the surface of the CTL, with what is generically referred to as an MHC-peptide complex located on the surface of the infected cell. MHC (major histocompatibility-complex)-encoded molecules have been subdivided into two types, and are referred to as class I and class II MHC-encoded molecules.

An attenuated strain of Mycobacterium bovis, Bacille Calmette-Guérin (BCG), is the only available vaccine today. The efficacy of this widely administered vaccine is a subject of controversy, varying from as high as 80% to as little as zero

The identification of peptides and proteins derived from M. tuberculosis that are effectively recognized by the cellular arm of the immune response forms the basis for a new and effective vaccine. Such peptides are displayed on the surface of infected cells in association with MHC class I and class II molecules and serve as recognition targets for cytolytic and helper T cells of the immune system.

In the human immune system, MHC molecules are referred to as human leukocyte antigens (HLA). Within the MHC, located on chromosome six, are three different genetic loci that encode for class I MHC molecules. MHC molecules encoded at these loci are referred to as HLA-A, HLA-B, and HLA-C. The genes that can be encoded at each of these loci are extremely polymorphic, and thus, different individuals within the population express different class I MHC molecules on the surface of their cells. HLA-A1, HLA-A2, HLA-A3, HLA-B7, and HLA-B8 are examples of different class I MHC molecules that can be expressed from these loci. The present disclosure involves peptides that are associated with the HLA-A2 molecules.

The peptides that associate with the MHC molecules can either be derived from proteins made within the cell, in which case they typically associate with class I MHC molecules (Rock, K. L. and Golde, U., Ann. Rev. Immunol., 17:739-779, (1999)) or they can be derived from proteins that are acquired from outside of the cell, in which case they typically associate with class II MHC molecules (Watts, C., Ann.Rev.lmmunol., 15:821-850, (1997)). The peptides that associate with a class I MHC molecule are typically nine amino acids in length, but can vary from a minimum length of eight amino acids to a maximum of fourteen amino acids in length. A class I MHC molecule with its bound peptide, or a class II MHC molecule with its bound peptide, is referred to as an MHC-peptide complex.

The process by which intact proteins are degraded into peptides is referred to as antigen processing. Two major pathways of antigen processing occur within cells (Rock, K. L. and Golde, U., Ann.Rev.Immunol., 17:739-779, (1999); Watts, C., Ann.Rev.Immunol., 15:821-850, (1997)). One pathway, which is largely restricted to cells that are antigen presenting cells such as dendritic cells, macrophages, and B cells, degrades proteins that are typically phagocytosed or endocytosed into the cell. Peptides derived from this pathway typically bind to class II MHC molecules. A second pathway of antigen processing is present in essentially all cells of the body.

This second pathway primarily degrades proteins that are made within the cells, and the peptides derived from this pathway primarily bind to class I MHC molecules. It is peptides from this second pathway of antigen processing that are referred to herein. Antigen processing by this latter pathway involves polypeptide synthesis and proteolysis in the cytoplasm. The peptides produced are then transported into the endoplasmic reticulum of the cell, associate with newly synthesized class I MHC molecules, and the resulting MHC-peptide complexes are then transported to the cell surface. Peptides derived from membrane and secreted proteins have also been identified. In some cases these peptides correspond to the signal sequence of the proteins that are cleaved from the protein by the signal peptidase. In other cases, it is thought that some fraction of the membrane and secreted proteins are transported from the endoplasmic reticulum into the cytoplasm where processing subsequently occurs.

Once bound to the class I MHC molecule and displayed on the surface of a cell, the peptides are recognized by antigen-specific receptors on CTLs. Mere expression of the class I MHC molecule itself is insufficient to trigger the CTL to kill the target cell if the antigenic peptide is not bound to the class I MHC molecule. Several methods have been developed to identify the peptides recognized by CTL, each method relying on the ability of a CTL to recognize and kill only those cells expressing the appropriate class I MHC molecule with the peptide bound to it (Rosenberg, S. A., Immunity, 10:281-287, (1999)). Such peptides can be derived from a non-self source, such as a pathogen (for example, following the infection of a cell by a bacterium, such as M. tuberculosis, or a virus) or from a self-derived protein within a cell, such as a cancerous cell.

Thus, in a typical scenario, macrophages phagocytize bacteria, process and degrade the bacterial proteins within the phagosome of the macrophage, and present them in association with MHC Class II molecules. However, this occurs in activated macrophages whereas early in tuberculosis infection, the macrophages are not activated and the phagocytized bacteria, here M. tuberculosis, live on and replicate in the phagosome vacuole. Such vacuoles are resistant to normal processing, such as lysosomal degradation of the vacuolar contents. Proteins secreted by these bacteria then exit the vacuoles through small pores in the vacuolar membrane into the cytoplasm where they enter the MHC Class I processing pathway, eventually being presented on the surface of the cell in association with MHC Class I, rather than Class II, molecules (Mazzaccaro, R. J. et al, PNAS, 93:11786 (1996), Teitelbaum, R et al, PNAS, 96:15190 (1999).

Different methodologies have typically been used for identifying the peptides that are recognized by CTLs, some of which suffer from various drawbacks. A useful technique has been the analysis of purified peptides by mass spectrometry. Fragmented masses are then analyzed for the peptide sequence and the database for the organism is analyzed and a hypothetical protein (i.e., an appropriate open reading frame) is identified containing the sequence of the peptide. The sequence can be confirmed by direct synthesis thereof (See Examples 4 and 5, below). Once prepared such sequences can be used to test their ability to activate CTLs against cells infected with the tubercle bacillus.

Immunization with bacterial-derived, class I MHC-encoded molecule-associated peptides, or with a precursor polypeptide or protein that contains the peptide, or with a gene that encodes a polypeptide or protein containing the peptide, are forms of immunotherapy that can be employed in the treatment of infections. These forms of immunotherapy require that immunogens be identified so that they can be formulated into an appropriate vaccine.

### BRIEF SUMMARY OF THE INVENTION

The present invention relates to Immunogens, such as polypeptides and functionally similar structures, comprising a novel epitopic peptide sequence of between 8 and 14, amino acids in length, most especially the sequence of SEQ ID NO: 1, 2, 3, 4 and 5 and which immunogens facilitate a cytotoxic T lymphocyte (CTL)-mediated immune response against bacterial infected cells, such as infected macrophages, especially macrophages infected with the tubercle bacillus.

Such immunogens do not include heat shock protein 65 (Hsp65) found in bacteria. The hsp of human and bacteria are homologous in certain regions but the peptide of the present invention comes from the regions unique to the bacteria. The portion of that protein at 15-20 amino acids in length would be an immunogenic peptide within the invention.

The present invention also relates to nucleic acid molecules that encode polypeptides comprising said epitopic peptide, and which can also be used to facilitate an immune response against tubercle infected cells.

The present invention provides compositions comprising the polypeptides and immunogens described herein whereby the oligopeptides and polypeptides of such immunogens are capable of inducing a CTL response against cells expressing a protein comprising an epitopic sequence of SEQ ID NO: 1, 2, 3, 4 and 5 presented in association with HLA-A2, a Class I MHC protein, which cells are infected with the tubercle bacillus, especially where these are infected macrophages.

In specific embodiments, the oligopeptides of the invention have a sequence that comprises SEQ ID NO: 1, 2, 3, 4 and 5, and are used as part of a larger structure, most advantageously a polypeptide, including both naturally occurring polypeptides and synthetic polypeptides. The immunogens of the invention incorporate such epitopic peptide sequences, either with such sequences attached to form a larger antigenic structure or just as part of a polypeptide sequence incorporating such peptides as part of the amino acid sequence thereof but not including heat shock protein 65 (HSP65).

Where the immunogens of the invention are polypeptides, or mixtures of polypeptides, such polypeptides can be of any length as long as part of their sequence comprises at least one peptide of SEQ ID NO: 1, 2, 3, 4 or 5, or sequence highly homologous thereto, ordinarily differing by no more than one amino acid residue, including multiple copies of said sequence, when it is desired to induce a CTL response against such peptide and thereby against tuberculosis (TB) infected cells, especially infected macrophages.

The present invention also provides methods that comprise contacting a lymphocyte, especially a CTL, with an immunogen, such as an immunogenic polypeptide, of the invention under conditions that induce a CTL response against a TB infected cell, especially a TB-infected macrophage. The methods of the invention contemplate contacting the CTL with the immunogenic peptide in vivo, in which case the peptides, polypeptides, and polynucleotides of the invention are used as vaccines, and are delivered as a pharmaceutical composition comprising a pharmaceutically acceptable carrier and the immunogen (typically along with an adjuvant or one or more cytokines).

Alternatively, the immunogens of the present invention can be used to induce a CTL response in vitro. The generated CTL can then be introduced into a patient with tuberculosis. Alternatively, the ability to generate CTLs in vitro can serve as a diagnostic for tuberculosis.

### DEFINITIONS

As used herein and except as noted otherwise, all terms are defined as given below.

The term “peptide” is used herein to designate a series of amino acid residues, connected one to the other typically by peptide bonds between the alpha-amino and carbonyl groups of the adjacent amino acids. The peptides are typically 9 amino acids in length, but can be as short as 8 amino acids in length, and as long as 14 amino acids in length.

The term “oligopeptide” is used herein to designate a series of amino acid residues, connected one to the other typically by peptide bonds between the alpha-amino and carbonyl groups of the adjacent amino acids. The length of the oligopeptide is not critical to the invention as long as the correct epitope or epitopes are maintained. The oligopeptides are typically less than about 30 amino acid residues in length, and greater than about 14 amino acids in length.

The term “polypeptide” designates a series of amino acid residues, connected one to the other typically by peptide bonds between the alpha-amino and carbonyl groups of the adjacent amino acids. The length of the polypeptide is not critical to the invention as long as the correct epitopes are maintained. In contrast to the terms peptide or oligopeptide, the term polypeptide is meant to refer to protein molecules of longer than about 30 residues in length.

A peptide, oligopeptide, protein, or polynucleotide coding for such a molecule is “immunogenic” (and thus an “immunogen” within the present invention) if it is capable of inducing an immune response. In the case of the present invention, immunogenicity is more specifically defined as the ability to induce a CTL-mediated response. Thus, an “immunogen” would be a molecule that is capable of inducing an immune response, and in the case of the present invention, a molecule capable of inducing a CTL response.

A T cell “epitope” is a short peptide molecule that binds to a class I or II MHC molecule and that is subsequently recognized by a T cell. T cell epitopes that bind to class I MHC molecules are typically 8-14 amino acids in length, and most typically 9 amino acids in length. T cell epitopes that bind to class II MHC molecules are typically 12-20 amino acids in length. In the case of epitopes that bind to class II MHC molecules, the same T cell epitope may share a common core segment, but differ in the length of the carboxy- and amino-terminal flanking sequences due to the fact that ends of the peptide molecule are not buried in the structure of the class II MHC molecule peptide-binding cleft as they are in the class I MHC molecule peptide-binding cleft.

As used herein, reference to a DNA sequence includes both single stranded and double stranded DNA. Thus, the specific sequence, unless the context indicates otherwise, refers to the single strand DNA of such sequence, the duplex of such sequence with its complement (double stranded DNA) and the complement of such sequence.

The term “coding region” refers to that portion of a gene which either naturally or normally codes for the expression product of that gene in its natural genomic environment, i.e., the region coding in vivo for the native expression product of the gene. The coding region can be from a normal, mutated or altered gene, or can even be from a DNA sequence, or gene, wholly synthesized in the laboratory using methods well known to those of skill in the art of DNA synthesis.

The term “nucleotide sequence” refers to a heteropolymer of deoxyribonucleotides. The nucleotide sequence encoding for a particular peptide, oligopeptide, or polypeptide may be naturally occurring or they may be synthetically constructed. Generally, DNA segments encoding the peptides, polypeptides, and proteins of this invention are assembled from cDNA fragments and short oligonucleotide linkers, or from a series of oligonucleotides, to provide a synthetic gene which is capable of being expressed in a recombinant transcriptional unit comprising regulatory elements derived from a microbial or viral operon.

The term “expression product” means that polypeptide or protein that is the natural translation product of the gene and any nucleic acid sequence coding equivalents resulting from genetic code degeneracy and thus coding for the same amino acid(s).

The term “fragment,” when referring to a coding sequence, means a portion of DNA comprising less than the complete coding region whose expression product retains essentially the same biological function or activity as the expression product of the complete coding region.

The term “DNA segment” refers to a DNA polymer, in the form of a separate fragment or as a component of a larger DNA construct, which has been derived from DNA isolated at least once in substantially pure form, i.e., free of contaminating endogenous materials and in a quantity or concentration enabling identification, manipulation, and recovery of the segment and its component nucleotide sequences by standard biochemical methods, for example, by using a cloning vector. Such segments are provided in the form of an open reading frame uninterrupted by internal non-translated sequences, or introns, which are typically present in eukaryotic genes. Sequences of non-translated DNA may be present downstream from the open reading frame, where the same do not interfere with manipulation or expression of the coding regions.

The term “primer” means a short nucleic acid sequence that is paired with one strand of DNA and provides a free 3′OH end at which a DNA polymerase starts synthesis of a deoxyribonucleotide chain.

The term “promoter” means a region of DNA involved in binding of RNA polymerase to initiate transcription.

The term “open reading frame (ORF)” means a series of triplets coding for amino acids without any termination codons and is a sequence (potentially) translatable into protein.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

The polynucleotides, and recombinant or immunogenic polypeptides, disclosed in accordance with the present invention may also be in “purified” form. The term “purified” does not require absolute purity; rather, it is intended as a relative definition, and can include preparations that are highly purified or preparations that are only partially purified, as those terms are understood by those of skill in the relevant art. For example, individual clones isolated from a cDNA library have been conventionally purified to electrophoretic homogeneity. Purification of starting material or natural material to at least one order of magnitude, preferably two or three orders, and more preferably four or five orders of magnitude is expressly contemplated. Furthermore, the claimed polypeptide which has a purity of preferably 0.001%, or at least 0.01% or 0.1%; and even desirably 1% by weight or greater is expressly contemplated.

The nucleic acids and polypeptide expression products disclosed according to the present invention, as well as expression vectors containing such nucleic acids and/or such polypeptides, may be in “enriched form.” As used herein, the term “enriched” means that the concentration of the material is at least about 2, 5, 10, 100, or 1000 times its natural concentration (for example), advantageously 0.01%, by weight, preferably at least about 0.1% by weight. Enriched preparations of about 0.5%, 1%, 5%, 10%, and 20% by weight are also contemplated. The sequences, constructs, vectors, clones, and other materials comprising the present invention can advantageously be in enriched or isolated form.

The term “active fragment” means a fragment that generates an immune response (i.e., has immunogenic activity) when administered, alone or optionally with a suitable adjuvant, to a mammal, especially a human, such immune response taking the form of stimulating a CTL response within the recipient animal, such as a human. Alternatively, the “active fragment” may also be used to induce a CTL response in vitro.

As used herein, the terms “portion,” “segment,” and “fragment,” when used in relation to polypeptides, refer to a continuous sequence of residues, such as amino acid residues, which sequence forms a subset of a larger sequence. For example, if a polypeptide were subjected to treatment with any of the common endopeptidases, such as trypsin or chymotrypsin, the oligopeptides resulting from such treatment would represent portions, segments or fragments of the starting polypeptide. This means that any such fragment will necessarily contain as part of its amino acid sequence a segment, fragment or portion, that is substantially identical, if not exactly identical, to the sequence of SEQ ID NO: 1, 2, 3, 4 or 5.

In accordance with the present invention, the sequences disclosed herein comprise the following: LMSLLSRV (SEQ ID NO: 1), GLIDIAPHQISSV (SEQ ID NO: 2), GLIDIAPHQISS (SEQ ID NO: 3), GLIDIAPHQI (SEQ ID NO: 4), and TLLQMPTL (SEQ ID NO: 5). For these sequences, the conventional one-letter amino acid codes are used. The corresponding three-letter codes are used in the accompanying sequence listing.

In accordance with the present invention, the term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:

Percent Identity=100 [1−(C/R)]

wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the herein above calculated Percent Identity is less than the specified Percent Identity.

The term “HSP65” refers to “heat shock protein 65” found in species of Mycobacteria as well as in human and mouse sources. These proteins exhibit varying degrees of sequence homology and represent an immunodominant antigenic site on the source cell.

### DETAILED SUMMARY OF THE INVENTION

The present invention relates generally to immunogens and immunogenic compositions, and methods of use therefor, for the prevention, treatment, and diagnosis of bacterial infections, especially tuberculosis. Disclosed according to the invention are immunogens comprising proteins or polypeptides whose amino acid sequences comprise one or more epitopic peptides with sequences homologous to, preferably identical to, the sequence of SEQ ID NO: 1, 2, 3, 4 and 5. The immunogens of the present invention expressly exclude Hsp65 protein from whatever source. For example, the sequence of SEQ ID NO: 5 is found in bovis HSP65. The peptides of SEQ ID NO: 2, 3 and 4 have been found in an M. tuberculosis protein dubbed “Rv0341.” The epitopic peptides (i.e., oligopeptides) of SEQ ID NO: 1, 2, 3, 4 and 5 have been found to be expressed by mammalian cell lines infected with M. tuberculosis. All are HLA type A2-associated.

In addition, the invention further relates to polynucleotides that can be used to stimulate a CTL response against bacterial-infected cells, especially cells infected with the causative organism of TB, most especially tubercle-infected macrophages.

In accordance with the present invention there are disclosed specific amino acid sequences (SEQ ID NO: 1, 2, 3, 4 and 5) which represent epitopic peptides (i.e. immunogenic peptide sequences) of at least about 8 amino acids in length and no longer than about 14 amino acids in length and which are present as part of a larger structure, such as a polypeptide or full length protein, to form an immunogen of the invention. Proteins present in the cells of M. tuberculosis show these sequences. In addition, synthetic oligopeptides and polypeptides according to the invention also contain this sequence in one or more copies.

When the immunogens of the present invention comprise, or are formed of, polypeptides, these have amino acid sequences that comprise at least one stretch, possibly two, three, four, or more stretches of about 8 to 14 residues in length and wherein any such segment within such sequence differs in amino acid sequence from the sequence of SEQ ID NO: 1, 2, 3, 4 or 5 by no more than about 1 amino acid residue, giving an overall sequence identity or homology of at least about 88%, preferably a conservative amino acid residue, especially amino acids of the same general chemical character, such as where they are hydrophobic amino acids, or polar amino acids, or acidic amino acids or basic (alkaline) amino acids. Such polypeptides expressly exclude Hsp65 protein itself (i.e., native Hsp65) from whatever source.

The present invention also relates to compositions comprising the immunogens and isolated peptides of the invention. For example, if an isolated peptide of 10 amino acids in length is used, alone or in a mixture with other peptides, such decapeptide may contain within its sequence a single stretch of 9 amino acid residues that contains at most one residue location that differs from the residue in the corresponding location of SEQ ID NO: 1 or 5 when said sequences are matched. An octapeptide would automatically differ by one residue from the nonapeptide sequence of SEQ ID NO: 1 or 5.

Peptides of the invention are commonly immunogens, or at least can have immunogenic activity, possibly requiring a larger carrier molecule to facilitate such activity, or said peptides may have immunogenic activity when part of a larger structure, such as a polypeptide, other than the protein found in the TB organism itself. Such peptides may also have immunogenic activity when part of a composition containing one or more of said epitopic peptides, which may be present in any combination and with each such peptide being present in one or more copies.

Said polypeptides can be of any desired length so long as they have immunogenic activity in that they are able, under a given set of desirable conditions, to elicit in vitro or in vivo the activation of cytotoxic T lymphocytes (CTLs) (i.e., a CTL response) against a presentation of a TB-infected cell, especially an infected macrophage, and when such proteins are presented along with MHC-1 proteins, such as where said proteins are presented in vitro or in vivo by an antigen presenting cell (APC). The proteins and polypeptides forming the immunogens of the present invention can be naturally occurring or may be synthesized chemically.

The epitopic sequence (SEQ ID NO: 1, 2, 3, 4 and 5) present within polypeptides and proteins forming the immunogens of the present invention include sequences as short as 7, preferably 8, amino acid residues and as long as 15, preferably 14, amino acids in length. The present invention also encompasses peptides at least about 88% identical to the peptides or sequences of SEQ ID NO: 1, 2, 3, 4 and 5 disclosed herein and to sequences differing from these sequences by no more than one amino acid, including fragments containing sequences having at least 8 residues in common with the sequences of SEQ ID NO: 1 and 5 over any nine residue length and wherein said homologous sequence of residues need not be continuous so that said length may contain up to one amino acid not in common with the sequence of SEQ ID NO: 1, 2, 3, 4 and 5 or be identical to said sequence but include one additional residue or have one less residue relative to said sequence and whereby such different amino acid unit or residue may occur anywhere within the corresponding stretch within said immunogen or polypeptide.

The present invention is also directed to an isolated polypeptide, including a purified polypeptide, especially one having immunogenic activity, the sequence of which comprises within it one or more copies of epitopic peptide sequences homologous, if not identical, to the sequence of SEQ ID NO: 1, 2, 3, 4 and/or 5 and wherein said sequences may differ by one amino acid residues from the sequence of SEQ ID NO: 1, 2, 3, 4 and 5. Thus, within the present invention, such polypeptide may contain as part of its amino acid sequence, oligopeptides having up to 8 amino acids in length and differing by no more than one amino acid residue as compared to the sequence of SEQ ID NO: 1, 2, 3, 4 and/or 5 such that the polypeptide comprises, in one specific embodiment, 2 segments each with a sequence differing by no more than one amino acid residue from SEQ ID NO: 1, 2, 3, 4 and/or 5 and 1 segment identical to SEQ ID NO: 1, 2, 3, 4 and/or 5. In other embodiments, other combinations and permutations of the epitopic sequence disclosed herein may be part of an immunogen of the present invention or of such a polypeptide so long as any such polypeptide comprises at least 2 such epitopes, whether such epitopes are identical or differ by a residue. In other preferred embodiments, such immunogen, especially where a polypeptide, may comprise as part of its amino acid sequence, a number of oligopeptide segments as disclosed herein such that there are 2, 3, 4, 5, or more such segments and wherein such segments are contiguous or are not contiguous or where some are contiguous and some are not contiguous.

The present invention further relates to isolated oligopeptides of at least 8 but not more than 14 amino acid units in length and having a sequence differing at most by no more than one amino acid residue from a sequence selected from the group consisting of the sequence of SEQ ID NO: 1, 2, 3, 4 and 5. Thus, the present invention relates to a immunogen comprising a peptide segment of at least 8 but not more than 14 amino acid units in length which segment comprises a sequence selected from the group consisting of the sequence of SEQ ID NO: 1, 2, 3, 4 and 5 or a sequence differing from said sequence by not more than 1 amino acid and wherein said immunogen is not hsp65 protein.

Where an isolated peptide or oligopeptide of the invention comprises a sequence identical to a sequence of SEQ ID NO: 1, 2, 3, 4 or 5, such oligopeptide may be one amino acid longer or shorter than said oligopeptide sequence. Thus, as a non-limiting example, an isolated oligopeptide within the present invention would include an isolated oligopeptide comprising a sequence of thirteen amino acid residues identical to the sequence of SEQ ID NO: 2 and further comprising an additional amino acid residue for a total of 14 residues in length, or an isolated oligonucleotide of 13 residues total length but differing from the sequence of SEQ ID NO: 2 by no more than one residue and an isolated oligopeptide of 12 amino acid residues in length and comprising the sequence of 12 residues derived from SEQ ID NO: 2, such as where said sequence is comprised of residues 1-12 or 2-13 of said sequence.

In a preferred embodiment, the isolated oligopeptide of the present invention are oligopeptides having an amino acid sequence selected from the group consisting of SEQ ID NO: 1, 2, 3, 4 and 5.

In preferred embodiments, where the isolated oligopeptides of the invention are homologous to the sequences of SEQ ID NO: 1, 2, 3, 4 and/or 5, said difference of one amino acid residue is the result of a substitution of one hydrophobic amino acid unit by another hydrophobic amino acid, or is the result of a substitution of one polar amino acid unit by another polar amino acid, or is a substitution of one acidic amino acid unit by another acidic amino acid, or is the result of a substitution of one basic amino acid unit by another basic amino acid.

The present invention further relates to a composition comprising one or more of the isolated oligopeptides of of the invention suspended in a pharmacologically acceptable carrier.

Oligopeptides as disclosed herein may themselves be prepared by methods well known to those skilled in the art. (Grant, G. A., Synthetic Peptides: A User's Guide, 1992, W. H. Freeman and Company, New York; Coligan, J. E. et al, Current Protocols in Protein Science, 1999, John Wiley & Sons, Inc., New York).

Besides the sequences of SEQ ID NO: 1, 2, 3, 4 and 5, the proteins and polypeptides forming the immunogens of the present invention may also comprise one or more other immunogenic amino acid stretches known to be associated with M. tuberculosis, and which may stimulate or enhance a CTL response whereby the immunogenic peptides associate with HLA-A2 or another class I MHC (i.e., MHC-1) molecule.

The immunogens of the present invention can be in the form of a composition of one or more of the different immunogens and wherein each immunogen is present in any desired relative abundance. Such compositions can be homogeneous or heterogeneous with respect to the individual immunogens or polypeptides of the invention, or the immunogenic peptide components present in such polypeptides or proteins or immunogens, having only one or more than one of such peptides. For example, an isolated peptide of the present invention can have the sequence of SEQ ID NO: 1, 2, 3, 4 and 5 or differ therefrom by 1 amino acid and such peptides can be used to form an immunogenic composition of said peptides as already disclosed herein.

The peptides, or oligopeptides, or polypeptides, useful in practicing the present invention may be derived by fractionation of naturally occurring proteins by methods such as protease treatment, or they may be produced by recombinant or synthetic methodologies that are well known and clear to the skilled artisan (Ausubel, F. M. et al, Current Protocols in Molecular Biology, 1999, John Wiley & Sons, Inc., New York; Coligan, J. E. et al, Current Protocols in Protein Science, 1999, John Wiley & Sons, Inc., New York; Molecular Cloning: A Laboratory Manual, 1989, Cold Spring Harbor Laboratory Press, Cold Spring Harbor). In obtaining an epitopic peptide of the invention, a human macrophage cell line was infected with an avirulent strain of M. tuberculosis. From lysates of the infected macrophages, MHC class I:peptide complexes were isolated by immunoafinity chromatography and peptides purified. As one example, from mass spectral analysis of the MHC associated peptides, the peptide LAASLLSRV (SEQ ID NO: 1), derived from the M. tuberculosis hypothetical protein Rv3808c, was identified. This peptide was subsequently shown to bind to the HLA-A2 molecule and is useful as an immunotherapeutic in the prevention and treatment of tuberculosis. The epitopic peptides or oligopeptides of SEQ ID NO: 2, 3 and 4 were obtained from the hypothetical protein Rv0341 produced by infected cells of cell line Mtb/U937 and that of SEQ ID NO: 5 from bovis Hsp65 by infected cells of cell line Mtb/THP1. All such peptides are HLA type A2.

Where the immunogen comprises two or more immunogenic epitopes, or epitopic peptides, they may be linked directly together, or through a spacer or linker, to form a larger structure, such as an oligopeptide, or polypeptide, or some other polymeric structure. The epitopic peptides may therefore be linked by any and all means that can be devised by the chemist so long as the immunogenic activity of the overall structure or complex is maintained or, at least, not reduced below a level useful for the methods of the invention (i.e., especially where said immunogenic activity comprises being capable of eliciting a CTL response).

Likewise, the immunogenic peptides disclosed herein may also be linked directly to, or through, a spacer or linker to: an immunogenic carrier such as serum albumin, tetanus toxoid, keyhole limpet hemocyanin, dextran, or a recombinant virus particle; an immunogenic peptide known to stimulate a T helper cell type immune response; a cytokine such as interferon gamma or GMCSF (Granulocyte-Monocyte Colony Stimulating Factor); a targeting agent such as an antibody or receptor ligand; a stabilizing agent such as a lipid; or a conjugate of a plurality of epitopes to a branched lysine core structure, such as the so-called “multiple antigenic peptide” described in (Posnett, D. N. et al., J.Biol.Chem., 263:1719-1725, (1988)); a compound such as polyethylene glycol to increase the half life of the peptide; or additional amino acids such as a leader or secretory sequence, or a sequence employed for the purification of the mature sequence.

Useful spacers and linkers are typically comprised of relatively small, neutral molecules, such as amino acids and which are substantially uncharged under physiological conditions. Such spacers are typically selected from the group of nonpolar or neutral polar amino acids, such as glycine, alanine, serine and other similar amino acids. Such optional spacers or linkers need not be comprised of the same residues and thus may be either homo- or hetero-oligomers. When present, such linkers will commonly be of length at least one or two, commonly 3, 4, 5, 6, and possibly as much as 10 or even up to 20 residues (in the case of amino acids). In addition, such linkers need not be composed of amino acids but any oligomeric structures will do as well so long as they provide the correct spacing so as to optimize the desired level of immunogenic activity of the immunogens of the present invention. The immunogen may therefore take any form that is capable of eliciting a CTL response.

In addition, the immunogenic peptides of the present invention may be part of an immunogenic structure via attachments other than conventional peptide bonds. Thus, any manner of attaching the peptides of the invention to an immunogen of the invention, such as an immunogenic polypeptide as disclosed herein, could provide an immunogenic structure as claimed herein. Thus, immunogens, such as proteins of the invention, are structures that contain the peptides disclosed according to the present invention but such immunogenic peptides may not necessarily be attached thereto by the conventional means of using ordinary peptide bounds. The immunogens of the present invention simply contain such peptides as part of their makeup, but how such peptides are to be combined to form the final immunogen is left to the talent and imagination of the user and is in no way restricted or limited by the disclosure contained herein.

The peptides that are naturally processed and bound to a class I MHC molecule in accordance with the invention need not be the optimal peptides for stimulating a CTL response. See, for example, (Parkhurst, M. R. et al., J.Immunol., 157:2539-2548, (1996); Rosenberg, S. A. et al., Nat.Med., 4:321-327, (1998)). Thus, there can be utility in modifying a peptide, such that it more readily induces a CTL response. Generally, peptides may be modified at two types of positions. The peptides may be modified at amino acid residues that are predicted to interact with the class I MHC molecule, in which case the goal is to create a peptide that has a higher affinity for the class I MHC molecule than does the parent peptide. The peptides can also be modified at amino acid residues that are predicted to interact with the T cell receptor on the CTL, in which case the goal is to create a peptide that has a higher affinity for the T cell receptor than does the parent peptide. Both of these types of modifications can result in a variant peptide that is related to a parent peptide, but which is better able to induce a CTL response than is the parent peptide. As used herein, the term “parent peptide” means an oligopeptide having the sequence of SEQ ID NO: 1, 2, 3, 4 and 5.

The parent peptides disclosed herein can be modified by the substitution of one or more residues at different, possibly selective, sites within the peptide chain. Such substitutions may be of a conservative nature, for example, where one amino acid is replaced by an amino acid of similar structure and characteristics, such as where a hydrophobic amino acid is replaced by another hydrophobic amino acid. Even more conservative would be replacement of amino acids of the same or similar size and chemical nature, such as where leucine is replaced by isoleucine. In studies of sequence variations in families of naturally occurring homologous proteins, certain amino acid substitutions are more often tolerated than others, and these are often show correlation with similarities in size, charge, polarity, and hydrophobicity between the original amino acid and its replacement, and such is the basis for defining “conservative substitutions.”

Conservative substitutions are herein defined as exchanges within one of the following five groups: Group 1—small aliphatic, nonpolar or slightly polar residues (Ala, Ser, Thr, Pro, Gly); Group 2—polar, negatively charged residues and their amides (Asp, Asn, Glu, Gln); Group 3—polar, positively charged residues (His, Arg, Lys); Group 4—large, aliphatic, nonpolar residues (Met, Leu, lie, Val, Cys); and Group 4—large, aromatic residues (Phe, Tyr, Trp). An acidic amino acid might also be substituted by a different acidic amino acid or a basic (i.e., alkaline) amino acid by a different basic amino acid.

Less conservative substitutions might involve the replacement of one amino acid by another that has similar characteristics but is somewhat different in size, such as replacement of an alanine by an isoleucine residue. Highly non-conservative replacements might involve substituting an acidic amino acid for one that is polar, or even for one that is basic in character. Such radical substitutions cannot, however, be dismissed as potentially ineffective since chemical effects are not totally predictable and radical substitutions might well give rise to serendipitous effects not otherwise predictable from simple chemical principles.

Of course, such substitutions may involve structures other than the common L-amino acids. Thus, D-amino acids might be substituted for the L-amino acids commonly found in the antigenic peptides of the invention and yet still be encompassed by the disclosure herein. In addition, amino acids possessing non-standard R groups (i.e., R groups other than those found in the common 20 amino acids of natural proteins) may also be used for substitution purposes to produce immunogens and immunogenic polypeptides according to the present invention.

If substitutions at more than one position are found to result in a peptide with substantially equivalent or greater antigenic activity as defined below, then combinations of those substitutions will be tested to determine if the combined substitutions result in additive or syngeneic effects on the antigenicity of the peptide. At most, no more than 1 position (possibly 2 positions) within the peptide would simultaneously be substituted.

Based on cytotoxicity assays, an epitope is considered substantially identical to the reference peptide if it has at least 10% of the antigenic activity of the reference peptide as defined by the ability of the substituted peptide to reconstitute the epitope recognized by a CTL in comparison to the reference peptide. Thus, when comparing the lytic activity in the linear portion of the effector:target curves with equimolar concentrations of the reference and substituted peptides, the observed percent specific killing of the target cells incubated with the substituted peptide should be equal to that of the reference peptide at an effector:target ratio that is no greater than 10-fold above the reference peptide effector:target ratio at which the comparison is being made.

Preferably, when the CTLs specific for an oligopeptide of the invention is tested against the substituted peptides, the peptide concentration at which the substituted peptides achieve half the maximal increase in lysis relative to background is no more than about 1 mM, preferably no more than about 1 μM, more preferably no more than about 1 nM, and still more preferably no more than about 100 pM, and most preferably no more than about 10 pM. It is also preferred that the substituted peptide be recognized by CTLs from more than one individual, at least two, and more preferably three individuals.

Thus, the epitopes of the present invention may be identical to naturally occurring tuberculosis-associated or tuberculosis-specific epitopes or may include epitopes that differ by up to 2 residues from the reference peptide, as long as they have substantially identical antigenic activity. The immunogenic peptides and polypeptides of the invention can be prepared synthetically, by recombinant DNA technology, or they can be isolated from natural sources such as bacilli expressing the parent protein product.

The polypeptides and oligopeptides disclosed herein can be synthesized in solution or on a solid support in accordance with conventional techniques. Various automated peptide synthesizers are commercially available and can be used in accordance with known protocols. See, for example, (Grant, G. A., Synthetic Peptides: A User's Guide, 1992, W. H. Freeman and Company, New York; Coligan, J. E. et al, Current Protocols in Protein Science, 1999, John Wiley & Sons, Inc., New York). Fragments of polypeptides of the invention can also be synthesized as intermediates in the synthesis of a larger polypeptide.

Recombinant DNA technology may be employed wherein a nucleotide sequence which encodes an immunogenic peptide or polypeptide of interest is inserted into an expression vector, transformed or transfected into an appropriate host cell, and cultivated under conditions suitable for expression. These procedures are well known in the art to the skilled artisan, as described in (Coligan, J. E. et al, Current Protocols in Immunology, 1999, John Wiley & Sons, Inc., New York; Ausubel, F. M. et al, Current Protocols in Molecular Biology, 1999, John Wiley & Sons, Inc., New York; Molecular Cloning: A Laboratory Manual, 1989, Cold Spring Harbor Laboratory Press, Cold Spring Harbor). Thus, recombinantly produced peptides or polypeptides can be used as the immunogens of the invention.

The coding sequences for peptides of the length contemplated herein can be synthesized on commercially available automated DNA synthesizers using protocols that are well know in the art. See for example, (Grant, G. A., Synthetic Peptides: A User's Guide, 1992, W. H. Freeman and Company, New York; Coligan, J. E. et al, Current Protocols in Protein Science, 1999, John Wiley & Sons, Inc., New York). The coding sequences can also be modified such that a peptide or polypeptide will be produced that incorporates a desired amino acid substitution. The coding sequence can be provided with appropriate linkers, be ligated into suitable expression vectors that are commonly available in the art, and the resulting DNA or RNA molecule can be transformed or transfected into suitable hosts to produce the desired fusion protein. A number of such vectors and suitable host systems are available, and their selection is left to the skilled artisan. For expression of the fusion proteins, the coding sequence will be provided with operably linked start and stop codons, promoter and terminator regions, and a replication system to provide an expression vector for expression in the desired host cell. For example, promoter sequences compatible with bacterial hosts are provided in plasmids containing convenient restriction sites for insertion of the desired coding sequence. The resulting expression vectors are transformed into suitable bacterial hosts. Of course, yeast, insect, and mammalian host cells may also be used, employing suitable vectors and control sequences.

Host cells are genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the genes of the present invention. The culture conditions, such as temperature, pH and the like, are those previously used with the host cell selected for expression, and will be apparent to the ordinarily skilled artisan.

More particularly, the present invention also includes recombinant constructs comprising one or more of the sequences as broadly described above. The constructs comprise a vector, such as a plasmid or viral vector, into which a sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. The following vectors are provided by way of example; Bacterial: pQE70, pQE60, pQE-9 (Qiagen), pBS, pD10, phagescript, psiX174, pBluescript SK, pBSKS, pNH8A, pNH16a, pNH18A, pNH46A (Stratagene); pTRC99a, pKK223-3, pKK233-3, pDR540, pRIT5 (Pharmacia); Eukaryotic: pWLNEO, pSV2CAT, pOG44, pXT1, pSG (Stratagene) pSVK3, pBPV, pMSG, pSVL (Pharmacia). However, any other plasmid or vector may be used as long as they are replicable and viable in the host.

In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Ausubel, F. M. et al, Current Protocols in Molecular Biology, 1999, John Wiley & Sons, Inc., New York; Molecular Cloning: A Laboratory Manual, 1989, Cold Spring Harbor Laboratory Press, Cold Spring Harbor). Such cells can routinely be utilized for assaying CTL activity by having said genetically engineered, or recombinant, host cells express the immunogenic peptides of the present invention.

Various mammalian cell culture systems can also be employed to express recombinant protein. Examples of mammalian expression systems include the COS-7 lines of monkey kidney fibroblasts, described by Gluzman, Cell, 23:175 (1981), and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide the required nontranscribed genetic elements.

The polypeptide can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the mature protein. Finally, high performance liquid chromatography (HPLC) can be employed for final purification steps.

The immunogenic peptides of the present invention may be used to elicit CTLs ex vivo from either healthy individuals or from patients with tuberculosis (or at risk thereof). Such responses are induced by incubating in tissue culture the individual's CTL precursor lymphocytes together with a source of antigen presenting cells and the appropriate immunogenic peptide. Examples of suitable antigen presenting cells include dendritic cells, macrophages, and activated B cells. Typically, the peptide at concentrations between 10 and 40 μg/ml, would be pre-incubated with the antigen presenting cells for periods ranging from 1 to 18 hrs. β₂-microglobulin (4 μg/ml) can be added during this time period to enhance binding. The antigen presenting cells may also be held at room temperature during the incubation period (Ljunggren, H. -G. et al., Nature, 346:476-480, (1990)) or pretreated with acid (Zeh, H. J., III et al., Hum.Immunol., 39:79-86, (1994)) to promote the generation of denatured class I MHC molecules which can then bind the peptide. The precursor CTLs (responders) are then added to the antigen presenting cells to which the immunogenic peptide has bound (stimulators) at responder to stimulator ratios of between 5:1 and 50:1, and most typically between 10:1 and 20:1. The co-cultivation of the cells is carried out at 37° C. in RPMI 1640, 10% fetal bovine serum, 2 mM L-glutamine, and IL-2 (5-20 Units/ml). Other cytokines, such as IL-1, IL-7, and IL-12 may also be added to the culture. Fresh IL-2-containing media is added to the cultures every 2-4 days, typically by removing one-half the old media and replenishing it with an equal volume of fresh media. After 7-10 days, and every 7-10 days thereafter, the CTL are restimulated with antigen presenting cells to which immunogenic peptide has been bound as described above. Fresh IL-2-containing media is added to the cells throughout their culture as described above. Three to four rounds of stimulation, and sometimes as many five to eight rounds of stimulation, are required to generate a CTL response that can then be measured in vitro. The above described protocol is illustrative only and should not be considered limiting. Many in vitro CTL stimulation protocols have been described and the choice of which one to use is well within the knowledge of the skilled artisan. The peptide-specific CTL can be further expanded to large numbers by treatment with anti-CD3 antibody. For example, see (Riddell, S. R. and Greenberg, P. D., J.Immunol.Methods, 128:189-201, (1990); Walter, E. A. et al., N.Engl.J.Med., 333:1038-1044, (1995)).

Antigen presenting cells that are to be used to stimulate a CTL response are typically incubated with peptide of an optimal length as disclosed herein that allows for direct binding of the peptide to the class I MHC molecule without additional processing. Larger oligopeptides and polypeptides are generally ineffective in binding to class I MHC molecules as they are not efficiently processed into an appropriately sized peptide in the extracellular milieu. There are a variety of approaches that are known in the art, however, that allow oligopeptides and polypeptides to be exogenously acquired by a cell, which then allows for their subsequent processing and presentation by a class I MHC molecule. Representative, but non-limiting examples of such approaches include electroporation of the molecules into the cell (Harding, C. H. III, Eur.J.Immunol., 22:1865-1869, (1992)), encapsulation of the molecules in liposomes which are fused to the cells of interest (Reddy, R. et al., J.Immunol.Methods, 141:157-163, (1991)), or osmotic shock in which the molecules are taken up via pinocytosis (Moore, M. W. et al., Cell, 54:777-785, (1988)). Thus, oligopeptides and polypeptides that comprise one or more of the peptides of the invention can be provided to antigen presenting cells in such a fashion that they are delivered to the cytoplasm of the cell, and are subsequently processed to allow presentation of the peptides.

Antigen presenting cells suitable for stimulating an in vitro CTL response that is specific for one or more of the peptides of the invention can also be prepared by introducing polynucleotide vectors encoding the sequences into the cells. These polynucleotides can be designed such that they express only a single peptide of the invention, multiple peptides of the invention, or even a plurality of peptides of the invention. There are a variety of approaches that are known in the art, that allow polynucleotides to be introduced and expressed in a cell, thus providing one or more peptides of the invention to the class I MHC molecule binding pathway. Representative, but non-limiting examples of such approaches include the introduction of plasmid DNA through particle-mediated gene transfer or electroporation (Tuting, T. et al., J.Immunol., 160:1139-1147, (1998)), or the transduction of cells with an adenovirus expressing the polynucleotide of interest (Perez-Diez, A. et al., Cancer Res., 58:5305-5309, (1998)). Thus, oligonucleotides that code for one or more of the peptides of the invention can be provided to antigen presenting cells in such a fashion that the peptides associate with class I MHC molecules and are presented on the surface of the antigen presenting cell, and consequently are available to stimulate a CTL response.

In specific embodiments, the methods of the present invention include a method for inducing a CTL response in vitro that is specific for an infected cell expressing HLA-A2, whereby the method comprises contacting a CTL precursor lymphocyte with an antigen presenting cell that has bound an immunogen comprising one or more copies of the peptides disclosed according to the invention.

In specific embodiments, the methods of the present invention include a method for inducing a CTL response in vitro that is specific for an infected cell expressing HLA-A2, whereby the method comprises contacting a CTL precursor lymphocyte with an antigen presenting cell that has exogenously acquired an immunogenic oligopeptide or polypeptide that comprises one or more copies of the peptides disclosed according to the invention.

A yet additional embodiment of the present invention is directed to a process for inducing a CTL response in vitro that is specific for an infected cell expressing HLA-A2, comprising contacting a CTL precursor lymphocyte with an antigen presenting cell that is expressing a polynucleotide coding for a polypeptide of the invention and wherein said polynucleotide is operably linked to a promoter.

A variety of techniques exist for assaying the activity of CTL. These techniques include the labeling of target cells with radionuclides such as Na₂⁵¹CrO₄ or ³H-thymidine, and measuring the release or retention of the radionuclides from the target cells as an index of cell death. Such assays are well-known in the art and their selection is left to the skilled artisan. Alternatively, CTLs are known to release a variety of cytokines when they are stimulated by an appropriate target cell, such as a cell expressing the relevant class I MHC molecule and the corresponding peptide. Non-limiting examples of such cytokines include IFN-γ, TNFα, and GM-CSF. Assays for these cytokines are well known in the art, and their selection is left to the skilled artisan. Methodology for measuring both target cell death and cytokine release as a measure of CTL reactivity are given in (Coligan, J. E. et al, Current Protocols in Immunology, 1999, John Wiley & Sons, Inc., New York).

After expansion of the antigen-specific CTLs, the latter are then adoptively transferred back into the patient, where they will destroy their specific target cell, especially macrophages infected with M. tuberculosis. The utility of such adoptive transfer is demonstrated in (North, R. J. et al., Infect.Immun., 67:2010-2012, (1999); Riddell, S. R. et al., Science, 257:238-241, (1992)). In determining the amount of cells to re-infuse, the skilled physician will be guided by the total number of cells available, the activity of the CTL as measured in vitro, and the condition of the patient. Preferably, however, about 1×10⁶ to about 1×10¹², more preferably about 1×10⁸ to about 1×10¹¹, and even more preferably, about 1×10⁹ to about 1×10¹⁰ peptide-specific CTL are infused. Methodology for re-infusing the T cells into a patient are well known and exemplified in U.S. Pat. No. 4,844,893 to Honski, et al., and U.S. Pat. No. 4,690,915 to Rosenberg.

The peptide-specific CTL can be purified from the stimulator cells prior to infusion into the patient. For example, monoclonal antibodies directed towards the cell surface protein CD8, present on CTL, can be used in conjunction with a variety of isolation techniques such as antibody panning, flow cytometric sorting, and magnetic bead separation to purify the peptide-specific CTL away from any remaining non-peptide specific lymphocytes or from the stimulator cells. These methods are well known in the art, and are their selection is left to the skilled artisan. It should be appreciated that generation of peptide-specific CTL in this manner, obviates the need for stimulating the CTL in the presence of tubercle-infected cells. Thus, there is no chance of inadvertently reintroducing infected cells into the patient.

Thus, one embodiment of the present invention relates to a process for treating a subject infected with TB characterized by macrophages expressing complexes of HLA-A2, whereby CTLs produced in vitro according to the present invention are administered in an amount sufficient to destroy the infected cells through direct lysis or to effect the destruction of the infected cells indirectly through the elaboration of cytokines.

Another embodiment of the present invention is directed to a process for treating a subject with tuberculosis characterized by infected cells, especially infected macrophages, expressing any class I MHC molecule and an epitope of SEQ ID NO: 1, 2, 3, 4 and 5, or a sequence highly homologous thereto, especially a sequence differing by no more than one amino acid unit from said epitope, whereby the CTLs are produced in vitro and are specific for the epitope or parent protein and are administered in an amount sufficient to destroy the infected cells through direct lysis or to effect the destruction of the infected cells indirectly through the elaboration of cytokines.

In additional embodiments, ex vivo generated CTLs can be used to identify and isolate the T cell receptor molecules specific for the peptide. The genes encoding the alpha and beta chains of the T cell receptor can be cloned into an expression vector system and transferred and expressed in naïve T cells from peripheral blood, T cells from lymph nodes, or T lymphocyte progenitor cells from bone marrow. These T cells, which would then be expressing a peptide-specific T cell receptor, would then have specific cytotoxic reactivity and could be used in adoptive therapy to destroy TB infected macrophages.

In addition to their use for therapeutic or prophylactic purposes, the immunogenic peptides of the present invention are useful as screening and diagnostic agents. Thus, the immunogenic peptides of the present invention, together with modem techniques of gene screening, make it possible to screen patients for the presence of genes encoding such peptides on cells obtained from patients suspected of TB infection and possibly at a much earlier date than otherwise presently available.

Alternatively, the immunogenic peptides disclosed herein, as well as functionally similar homologs thereof, may be used to screen a sample for the presence of CTLs that specifically recognize the corresponding epitopes. The lymphocytes to be screened in this assay will normally be obtained from the peripheral blood, but lymphocytes can be obtained from other sources, including lymph nodes, spleen, and pleural fluid. The peptides of the present invention may then be used as a diagnostic tool to evaluate the efficacy of the immunotherapeutic treatments disclosed herein. Thus, the in vitro generation of CTLs as described above would be used to determine if patients are likely to respond to the peptide in vivo. Similarly, the in vitro generation of CTLs could be done with samples of lymphocytes obtained from the patient before and after treatment with the peptides and other immunogens of the invention. Successful generation of CTLs in vivo should then be recognized by a correspondingly easier ability to generate peptide-specific CTLs in vitro from lymphocytes obtained following treatment in comparison to those obtained before treatment.

The oligopeptides of the invention, such as SEQ ID NO: 1, 2, 3, 4 or 5, can also be used to prepare class I MHC tetramers which can be used in conjunction with flow cytometry to quantitate the frequency of peptide-specific CTL that are present in a sample of lymphocytes from an individual. Specifically, for example, class I MHC molecules comprising HLA-A2 and peptides highly homologous, meaning differing by 1 amino acid residue, including where, for example, the peptide sequence has 8 or 10 residues, to SEQ ID NO:1 would be combined to form tetramers as exemplified in U.S. Pat. No. 5,635,363. Said tetramers would find use in monitoring the frequency of CTLs specific for the combination of HLA-A2 and a peptide of SEQ ID NO:1 in the peripheral blood or lymph nodes an individual undergoing immunotherapy with the peptides, proteins, or polynucleotides of the invention, and it would be expected that successful immunization would lead to an increase in the frequency of the peptide-specific CTLs.

As stated above, a vaccine in accordance with the present invention may include one or more of the hereinabove described polypeptides or active fragments thereof, or a composition, or pool, of immunogenic peptides disclosed herein. When employing more than one polypeptide or active fragment, such as two or more polypeptides and/or active fragments may be used as a physical mixture or as a fusion of two or more polypeptides or active fragments. The fusion fragment or fusion polypeptide may be produced, for example, by recombinant techniques or by the use of appropriate linkers for fusing previously prepared polypeptides or active fragments.

The immunogenic molecules of the invention, including vaccine compositions, may be utilized according to the present invention for purposes of preventing, suppressing or treating diseases causing the expression of the immunogenic peptides disclosed herein, such as where the antigen is being expressed by TB infected cells. As used in accordance with the present invention, the term “prevention” relates to a process of prophylaxis in which an animal, especially a mammal, and most especially a human, is exposed to an immunogen of the present invention prior to the induction or onset of the disease process. Thus, the immunogen could be administered to the general population as is frequently done for infectious diseases. Alternatively, the term “suppression” is often used to describe a condition wherein the disease process has already begun but obvious symptoms of said condition have yet to be realized. Thus, the cells of an individual may have become infected but no outside signs of the disease have yet been clinically recognized. In either case, the term prophylaxis can be applied to encompass both prevention and suppression. Conversely, the term “treatment” is often utilized to mean the clinical application of agents to combat an already existing condition whose clinical presentation has already been realized in a patient. This would occur where an individual has already been diagnosed as having a tuberculosis.

It is understood that the suitable dosage of an immunogen of the present invention will depend upon the age, sex, health, and weight of the recipient, the kind of concurrent treatment, if any, the frequency of treatment, and the nature of the effect desired. However, the most preferred dosage can be tailored to the individual subject, as determined by the researcher or clinician. The total dose required for any given treatment will commonly be determined with respect to a standard reference dose as set by a manufacturer, such as is commonly done with vaccines, such dose being administered either in a single treatment or in a series of doses, the success of which will depend on the production of a desired immunological result (i.e., successful production of a CTL-mediated response to the antigen, which response gives rise to the prevention and/or treatment desired). Thus, the overall administration schedule must be considered in determining the success of a course of treatment and not whether a single dose, given in isolation, would or would not produce the desired immunologically therapeutic result or effect.

The therapeutically effective amount of a composition containing one or more of the immunogens of this invention, is an amount sufficient to induce an effective CTL response to the antigen and to cure or arrest disease progression. Thus, this dose will depend, among other things, on the identity of the immunogens used, the nature of the disease condition, the severity of the disease condition, the extent of any need to prevent such a condition where it has not already been detected, the manner of administration dictated by the situation requiring such administration, the weight and state of health of the individual receiving such administration, and the sound judgment of the clinician or researcher. Thus, for purposes of prophylactic or therapeutic administration, effective amounts would generally lie within the range of from 1.0 μg to about 5,000 μg of peptide for a 70 kg patient, followed by boosting dosages of from about 1.0 μg to about 1,000 μg of peptide pursuant to a boosting regimen over days, weeks or even months, depending on the recipient's response and as necessitated by subsequent monitoring of CTL-mediated activity within the bloodstream. Of course, such dosages are to be considered only a general guide and, in a given situation, may greatly exceed such suggested dosage regimens where the clinician believes that the recipient's condition warrants more a aggressive administration schedule. Needless to say, the efficacy of administering additional doses, and of increasing or decreasing the interval, may be re-evaluated on a continuing basis, in view of the recipient's immunocompetence.

For such purposes, the immunogenic compositions according to the present invention may be used against a disease condition such as tuberculosis by administration to an individual by a variety of routes. The composition may be administered parenterally or orally, and, if parenterally, either systemically or topically. Parenteral routes include subcutaneous, intravenous, intradermal, intramuscular, intraperitoneal, intranasal, transdermal, or buccal routes. One or more such routes may be employed. Parenteral administration can be, for example, by bolus injection or by gradual perfusion over time.

Generally, vaccines are prepared as injectables, in the form of aqueous solutions or suspensions. Vaccines in an oil base are also well known such as for inhaling. Solid forms which are dissolved or suspended prior to use may also be formulated. Pharmaceutical carriers, diluents and excipients are generally added that are compatible with the active ingredients and acceptable for pharmaceutical use. Examples of such carriers include, but are not limited to, water, saline solutions, dextrose, or glycerol. Combinations of carriers may also be used. These compositions may be sterilized by conventional, well known sterilization techniques including sterile filtration. The resulting solutions may be packaged for use as is, or the aqueous solutions may be lyophilized, the lyophilized preparation being combined with sterile water before administration. Vaccine compositions may further incorporate additional substances to stabilize pH, or to function as adjuvants, wetting agents, or emulsifying agents, which can serve to improve the effectiveness of the vaccine.

The concentration of the CTL stimulatory peptides of the invention in pharmaceutical formulations are subject to wide variation, including anywhere from less than 0.01% by weight to as much as 50% or more. Factors such as volume and viscosity of the resulting composition must also be considered. The solvents, or diluents, used for such compositions include water, possibly PBS (phosphate buffered saline), or saline itself, or other possible carriers or excipients.

The immunogens of the present invention may also be contained in artificially created structures such as liposomes, ISCOMS, slow-releasing particles, and other vehicles which increase the immunogenicity and/or half-life of the peptides or polypeptides in serum. Liposomes include emulsions, foams, micelies, insoluble monolayers, liquid crystals, phospholipid dispersions, lamellar layers and the like. Liposomes for use in the invention are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by (Coligan, J. E. et al, Current Protocols in Protein Science, 1999, John Wiley & Sons, Inc., New York) and see also U.S. Pat. Nos. 4,235,871, 4,501,728, 4,837,028, and 5,019,369.

Liposomes containing the peptides or polypeptides of the invention can be directed to the site of lymphoid cells where the liposomes then deliver the selected immunogens directly to antigen presenting cells. Targeting can be achieved by incorporating additional molecules such as proteins or polysaccharides into the outer membranes of said structures, thus resulting in the delivery of the structures to particular areas of the body, or to particular cells within a given organ or tissue.

The immunogens of the present invention may also be administered as solid compositions. Conventional nontoxic solid carriers including pharmaceutical grades of mannitol, lactose, starch, magnesium, cellulose, glucose, sucrose, sodium saccharin, and the like. Such solid compositions will often be administered orally, whereby a pharmaceutically acceptable nontoxic composition is formed by incorporating the peptides and polypeptides of the invention with any of the carriers listed above. Generally, such compositions will contain 10-95% active ingredient, and more preferably 25-75% active ingredient.

Aerosol administration is also an alternative, requiring only that the immunogens be properly dispersed within the aerosol propellant. Typical percentages of the peptides or polypeptides of the invention are 0.01%-20% by weight, preferably 1%-10%. The use of a surfactant to properly disperse the immunogen may be required. Representative surfactants include the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1-20% by weight of the composition, preferably 0.25-5%. Typical propellants for such administration may include esters and similar chemicals but are by no means limited to these. A carrier, such as lecithin for intranasal delivery, may also be included.

The peptides and polypeptides of the invention may also be delivered with an adjuvant. Adjuvants include, but are not limited to complete or incomplete Freund's adjuvant, Montanide ISA-51, aluminum phosphate, aluminum hydroxide, alum, and saponin. Adjuvant effects can also be obtained by injecting a variety of cytokines along with the immunogens of the invention. These cytokines include, but are not limited to IL-1, IL-2, IL-7, IL-12, and GM-CSF.

The peptides and polypeptides of the invention can also be added to professional antigen presenting cells such as dendritic cells that have been prepared ex vivo. For example, the dendritic cells could be prepared from CD34 positive stem cells from the bone marrow, or they could be prepared from CD14 positive monocytes obtained from the peripheral blood. The dendritic cells are generated ex vivo using cytokines such as GM-CSF, IL-3, IL4, TNF, and SCF. The cultured DC are then pulsed with peptides at various concentrations using standard methods that are well known in the art. The peptide-pulsed dendritic cells can then be administered either intraveneously, subcutaneously, or intradermally, and the immunization may also include cytokines such as IL-2 or IL-12.

The present invention is also directed to a vaccine in which an immunogen of the present invention is delivered or administered in the form of a polynucleotide encoding the a polypeptide or active fragment as disclosed herein, whereby the peptide or polypeptide or active fragment is produced in vivo. The polynucleotide may be included in a suitable expression vector and combined with a pharmaceutically acceptable carrier. For example, the peptides or polypeptides could be expressed in plasmid DNA and nonreplicative viral vectors such as vaccinia, fowlpox, Venezuelan equine encephalitis virus, adenovirus, or other RNA or DNA viruses. These examples are meant to be illustrative only and should not be viewed as self-limiting A wide variety of other vectors are available and are apparent to those skilled in the art from the description given herein. In this approach, a portion of the nucleotide sequence of the viral vector is engineered to express the peptides or polypeptides of the invention. Vaccinia vectors and methods useful in immunization protocols are described in U.S. Pat. No. 4,722,848, the disclosure of which is incorporated herein by reference in its entirety.

Regardless of the nature of the composition given, additional therapeutic agents may also accompany the immunogens of the present invention. Thus, for purposes of treating tuberculosis, compositions containing the immunogens disclosed herein may, in addition, contain other anti-tubercle pharmaceuticals.

The present invention also relates to antibodies that react with immunogens, such as a polypeptide comprising one or more of the epitopic peptides of SEQ ID NO: 1-5 as disclosed herein. Active fragments of such antibodies are also specifically contemplated. Such antibodies, and active fragments of such antibodies, for example, and Fab structure, may react with, including where it is highly selective or specific for, an immunogenic structure comprising 2, 3, 4 or more of the epitopic peptides of the invention.

With the advent of methods of molecular biology and recombinant technology, it is now possible to produce antibody molecules by recombinant means and thereby generate gene sequences that code for specific amino acid sequences found in the polypeptide structure of the antibodies. Such antibodies can be produced by either cloning the gene sequences encoding the polypeptide chains of said antibodies or by direct synthesis of said polypeptide chains, with in vitro assembly of the synthesized chains to form active tetrameric (H₂L₂) structures with affinity for specific epitopes and antigenic determinants. This has permitted the ready production of antibodies having sequences characteristic of neutralizing antibodies from different species and sources.

Regardless of the source of the antibodies, or how they are recombinantly constructed, or how they are synthesized, in vitro or in vivo, using transgenic animals, such as cows, goats and sheep, using large cell cultures of laboratory or commercial size, in bioreactors or by direct chemical synthesis employing no living organisms at any stage of the process, all antibodies have a similar overall 3 dimensional structure. This structure is often given as H₂L₂ and refers to the fact that antibodies commonly comprise 2 light (L) amino acid chains and 2 heavy (H) amino acid chains. Both chains have regions capable of interacting with a structurally complementary antigenic target. The regions interacting with the target are referred to as “variable” or “V” regions and are characterized by differences in amino acid sequence from antibodies of different antigenic specificity.

The variable regions of either H or L chains contains the amino acid sequences capable of specifically binding to antigenic targets. Within these sequences are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure.

The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed L1, L2, L3, H1, H2, H3) for the respective light (L) and heavy (H) chains. The accepted CDR regions have been described by Kabat et al, J. Biol. Chem. 252:6609-6616 (1977).

In all mammalian species, antibody polypeptides contain constant (i.e., highly conserved) and variable regions, and, within the latter, there are the CDRs and the so-called “framework regions” made up of amino acid sequences within the variable region of the heavy or light chain but outside the CDRs.

The antibodies disclosed according to the invention may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain. Such antibodies may also include fragments, such as Fab and F(ab₂)′ fragments, capable of reacting with and binding to any of the polypeptides disclosed herein as being receptors.

In addition, the immunogens of the present invention can be used to stimulate the production of antibodies for use in passive immunotherapy, for use as diagnostic reagents, and for use as reagents in other processes such as affinity chromatography.

In one embodiment, the present invention relates to a process for treating an animal, such as a human patient, afflicted with tuberculosis characterized by tuberculosis infected cells expressing HLA-A2, comprising administering to said patient an effective amount of an antibody as disclosed herein in a pharmaceutically acceptable carrier. Such antibody reacts with, or is specific or selective for, an immunogen comprising one or more of the epitopic peptides of the invention.

In another embodiment, the present invention relates to a process for protecting an animal, such as a human patient, against infection with tuberculosis characterized by tuberculosis infected cells expressing HLA-A2, comprising administering to an animal, such as a human patient, at risk of such infection, an effective amount of an antibody as disclosed herein in a pharmaceutically acceptable carrier. Such antibody reacts with, or is specific or selective for, an immunogen comprising one or more of the epitopic peptides of the invention.

A specific embodiment of the present invention relates to a method for inducing a CTL response in a subject, wherein the immunogen is in the form of one or more peptides. The method comprises administering to subjects that express HLA-A2, at least one CTL epitope, wherein said epitope or epitopes are selected from a group comprising the peptides disclosed according to the invention, in an amount sufficient to induce a CTL response to infected macrophages expressing HLA-A2.

While the below examples are provided to illustrate the invention, it is to be understood that these methods and examples in no way limit the invention to the embodiments described herein and that other embodiments and uses will no doubt suggest themselves to those skilled in the art. All publications, patents, and patent applications cited herein are hereby incorporated by reference, as are the references cited therein. It is also to be understood that throughout this disclosure where the singular is used, the plural may be inferred and vice versa and use of either is not to be considered limiting. It should be borne in mind that although these examples recite specific oligopeptide sequences of the invention, as well as specific cell lines, the methodology disclosed in the examples applies equally, with any obvious modifications, to use of the other oligopeptides and cell lines disclosed herein according to the present invention.

### EXAMPLE 1

### Cell Line and Infection with M. tuberculosis

A variant of the human macrophage cell line U937 expressing the HLA-A2 molecule (U937/A2) (Wuorela, M., et al. Infect and Immun., 65:2060 (1997) was grown in spinner bottles in RPMI1640 supplemented with 10% fetal bovine serum, 2 mM L-glutamine, 1 mM HEPES and 300 g/ml G418. Cells were treated with 20 ng/ml phorbol myristic acetate (PMA) for 24 hr to enhance phagocytosis. PMA treated cells were the infected with an avirulent strain of M. tuberculosis (H57Ra) at a multiplicity of infection of 5 cfu/cell. Twenty-four hr after infection, cells were harvested, washed two times in phosphate buffered saline (pH 7.4) and cell pellets stored at −80° C.

### EXAMPLE 2

### Immunoaffinity Purification

Frozen, infected cell pellets were solubilized at 5-10×10⁶ cells/ml in 20 mM Tris, pH 8.0, 150 mM NaCl, 1% CHAPS, 18.5 μg/ml iodoacetamide, 5 μg/ml aprotonin, 10 μg/ml leupeptin, 10 μg/ml pepstatin A, 5 mM EDTA, 0.2% sodium azide, and 17.4 μg/ml phenylmethylsulfonyl fluoride for 1 h. This and all subsequent steps were performed with ice-cold solutions and at 4° C. The lysates were then centrifuged at 100,000× g, the pellets discarded, and the supematants passed through a 0.22 μm filter. The supematants were then passed over a series of columns with the first containing Sepharose, and the second containing the HLA-A2-specific monoclonal antibody BB7.2, bound to a protein A-Sepharose matrix. The second column was then sequentially washed with 20 column volumes of 20 mM Tris, pH 8.0, 150 mM NaCl, 20 column volumes of 20 mM Tris, pH 8.0, 1.0 M NaCl, and 20 column volumes of 20 mM Tris, pH 8.0. The peptides were eluted from the column with 5 column volumes of 10% acetic acid. The isolated HLA-A2 molecules were then boiled for 5 min to further dissociate any bound peptide from the heavy chains. The peptides were then separated from the co-purifying class I heavy chain and β₂-microglobulin by centrifugation on a Ultrafree-CL membrane with a nominal molecular weight cut-off of 5,000 Daltons (Millipore, Beford, Mass.).

### EXAMPLE 3

### Peptide Fractionation

The peptide extracts were fractionated by RP-HPLC (Reverse Phase—High Performance Liquid Chromatography) using an Applied Biosystems (ABI) model 140B system. The extracts were concentrated by vacuum centrifugation from about 20 ml down to 250 μl and injected onto a Higgins (Mountain View, Calif.) C18 Haisil column (2.1 mm×4 cm; 300 Å; 5 μm). The peptides were eluted using a gradient of acetonitrile/0.085% TFA (trifluoroacetic acid) in 0.1% TFA/water, with the concentration of acetonitrile increasing from 0-9% (0-5 minutes), 9-36% (5-55 minutes), and 36-60% (55-62 minutes).

### EXAMPLE 4

### Identification of Peptides that Associate with HLA-A2

To identify the peptides associated with the HLA-A2 molecule present on the surface of the macrophage U937/A2 cell line, purified peptides were loaded onto a reverse phase microcapillary column and gradient eluted through an electrospray interface directly into a quadrupole ion trap mass spectrometer. Analysis of the fragmented masses generated from the collision-activated dissociation (CAD) of the selected peptide molecular ions allowed the determination of the peptide sequence as LAASLLSRV (SEQ ID NO:1). Searching the M. tuberculosis genome database (Sanger Center) revealed the source of the peptide LMSLLSRV as Rv3808c, a hypothetical protein encoded by an open reading frame within the M. tuberculosis genome.

### EXAMPLE 5

### Confirmation of the Peptide Sequence

Peptides were synthesized using a Gilson (Madison, Wis.) AMS 422 multiple peptide synthesizer. Ten μmol quantities were synthesized using conventional FMOC amino acids, resins and chemical techniques. Peptides were purified by RP-HPLC using a 4.6 mm×100 mm POROS (Perseptive Biosystems, Cambridge, Mass.) column and a 10 min, 0-60% acetonitrile in 0.1% TFA gradient. The CAD mass spectra of a synthetic peptide corresponding to SEQ ID NO:1 and the chromatographic co-elution of the synthetic and unknown peptides unequivocally identified the unknown as having the sequence of SEQ ID NO:1. The same or similar procedures were used to identify the other sequences disclosed herein.

The peptides of SEQ ID NO: 2 (GLIDIAPHQISSV), 3 (GLIDIAPHQISS), and 4 (GLIDIAPHQI) are derived from a hypothetical protein of the M. tuberculosis genome (and are thus from the same protein). The peptide of SEQ ID NO: 5 (TLLQMPTL) is from M. tuberculosis and bovis hsp56 infected THP1 cells.

What is claimed is:

1. An immunogen comprising a peptide segment of at least 8 but not more than 14 amino acid units in length which segment comprises a sequence selected from the group consisting of the sequence of SEQ ID NO: 1, 2, 3, 4 and 5 or a sequence differing from said sequence by not more than 1 amino acid and wherein said immunogen is not hsp65 protein.

2. The immunogen of claim 1 wherein said peptide segment has an amino acid sequence selected from the group consisting of SEQ ID NO: 1, 2, 3, 4 and 5.

3. The immunogen of claim 1 or 2 wherein said immunogen segment comprises at least 5 copies of one or more of said peptides.

4. The immunogen of claim 1 or 2 wherein said immunogen is a polypeptide.

5. The immunogen of claim 1 wherein said difference of one amino acid residue is the result of a substitution of one hydrophobic amino acid unit by another hydrophobic amino acid.

6. The immunogen of claim 1 wherein said difference of one amino acid residue is the result of a substitution of one polar amino acid unit by another polar amino acid.

7. The immunogen of claim 1 wherein said difference of one amino acid residue is the result of a substitution of one acidic amino acid unit by another acidic amino acid.

8. The immunogen of claim 1 wherein said difference of one amino acid residue is the result of a substitution of one basic amino acid unit by another basic amino acid.

9. A polynucleotide comprising a polynucleotide sequence encoding a polypeptide according to claims 4.

10. The polynucleotide of claim 9 wherein said polynucleotide is DNA.

11. The polynucleotide of claim 9 wherein said polynucleotide is RNA.

12. A vector comprising a polynucleotide of claim 9.

13. A recombinant mammalian cell comprising the vector of claim 12 and expressing said polynucleotide.

14. A vaccine composition comprising an immunologically active amount of the immunogen of claim 1, 2, 3, 4, 5, 6, 7 or 8 wherein said immunogen is suspended in a pharmaceutically acceptable carrier.

15. An antibody specific for an immunogen of claim 1, 2, 3, 4, 5, 6, 7 or 8.

16. A process for inducing a cytotoxic T lymphocyte (CTL) in vitro that is specific for a tuberculosis infected cell expressing HLA-A2 comprising contacting a precursor CTL with an immunogen of claim 1 under conditions that generate a CTL response to such an infected cell.

17. A process for inducing a cytotoxic T lymphocyte (CTL) in vitro that is specific for a tuberculosis infected cell expressing HLA-A2 comprising contacting a precursor CTL with an immunogen of claim 2 under conditions that generate a CTL response to such an infected cell.

18. A process for inducing a CTL response in vitro that is specific for a tuberculosis infected cell expressing HLA-A2, said process comprising contacting a precursor CTL with a mammalian cell of claim 13.

19. A process for treating a subject with tuberculosis characterized by tuberculosis infected cells expressing HLA-A2, said process comprising administering CTLs induced by the processes of claims 16, 17 or 18 in an amount sufficient to destroy the infected cells through direct lysis or to effect the destruction of the infected cells indirectly through the elaboration of cytokines.

20. The process of claim 18 wherein said infected cells are macrophages.

21. A process for treating a tuberculosis-afflicted subject characterized by cells expressing any class I MHC molecule and a gene coding for an epitopic peptide sequence of SEQ ID NO: 1, 2, 3, 4 or 5, whereby the CTLs of claim 19 are administered in an amount sufficient to destroy the infected cells through direct lysis or to effect the destruction of the infected cells indirectly through the elaboration of cytokines.

22. The process for claim 21 wherein said infected cells are macrophages.

23. A process for inducing a CTL response in a subject, said process comprising administering at least one immunogen of claim 1, 2, 3, 4, 5, 6, 7 or 8, including combinations thereof, to an HLA-A2 positive subject and in an amount sufficient to induce a CTL response to tuberculosis-infected cells expressing HLA-A2.

24. An isolated peptide of at least 8 but not more than 14 amino acid units in length and having a sequence differing by no more than one amino acid residue from a sequence selected from the group consisting of the sequence of SEQ ID NO: 1,2,3,4 and 5.

25. The isolated peptide of claim 24 wherein said oligopeptide has an amino acid sequence selected from the group consisting of SEQ ID NO: 1, 2, 3, 4 and 5.

26. The isolated peptide of claim 24 or 25 wherein said difference of one amino acid residue is the result of a substitution of one hydrophobic amino acid unit by another hydrophobic amino acid.

27. The isolated peptide of claim 24 or 25 wherein said difference of one amino acid residue is the result of a substitution of one polar amino acid unit by another polar amino acid.

28. The isolated peptide of claim 24 or 25 wherein said difference of one amino acid residue is the result of a substitution of one acidic amino acid unit by another acidic amino acid.

29. The isolated peptide of claim 24 or 25 wherein said difference of one amino acid residue is the result of a substitution of one basic amino acid unit by another basic amino acid.

30. A composition comprising one or more of the isolated peptides of claim 24 or 25 suspended in a pharmacologically acceptable carrier.

31. A process for treating a patient afflicted with tuberculosis characterized by tuberculosis infected cells expressing HLA-A2, comprising administering to said patient an effective amount of the antibody of claim 15 in a pharmaceutically acceptable carrier.

32. A process for protecting a patient against infection with tuberculosis characterized by tuberculosis infected cells expressing HLA-A2, comprising administering to a patient at risk of such infection an effective amount of the antibody of claim 15 in a pharmaceutically acceptable carrier.


SEQUENCE LISTING (5 sequences)

SEQ ID NO: 1 - protein, 9 aa, Mycobacterium tuberculosis
LAASLLSRV

SEQ ID NO: 2 - protein, 13 aa, Mycobacterium tuberculosis
GLIDIAPHQISSV

SEQ ID NO: 3 - protein, 12 aa, Mycobacterium tuberculosis
GLIDIAPHQISS

SEQ ID NO: 4 - protein, 10 aa, Mycobacterium tuberculosis
GLIDIAPHQI

SEQ ID NO: 5 - protein, 9 aa, Mycobacterium tuberculosis
TLLQAAPTL